(19) United States
(12) Patent Application Publication
(10) Pub. No.: US 2010/0105634 A1
(43) Pub. Date: Apr. 29, 2010

(54) USE OF GLUCOSAMINE AS A MENTAL AND PHYSICAL STRESS RECOVERY ENHANCER AND A PERFORMANCE ENHANCER

(75) Inventors: John Andrew Bohlmann, Ottumwa, IA (US); Elizabeth Ann Dierickx, Minneapolis, MN (US); Brent Daniel Rogers, Ottumwa, IA (US)

Correspondence Address: CARGILL, INCORPORATED, P.O. Box 5624, MINNEAPOLIS, MN, 55440-5624, US

(73) Assignee: CARGILL, INCORPORATED, Wayzata, MN (US)

(21) Appl. No.: 12/529,978

(22) PCT Filed: Mar. 7, 2008
(86) PCT No.: PCT/US2008/056179

§ 371 Date: Sep. 4, 2009

Related U.S. Application Data
(60) Provisional application No. 60/905,559, filed on Mar. 7, 2007.

(51) Int. Cl. A61K 31/7008; C07H 5/06; A61P 25/00

(52) U.S. Cl. 514/62; 536/55.2

(57) ABSTRACT

The present invention provides a composition comprising glucosamine salts or derivatives for rapid enhancement of at least one of mental performance and physical performance of a subject. The composition enhances the mental performance and physical performance by reducing at least one kind of cellular stress effects in the body of the subject. In one embodiment mental performance is enhanced by improving memory recall of the subject. The composition according to an embodiment of the present invention is an ingestion product.

### PRIORITY CLAIM

This application claims the benefit of U.S. Provisional Application No. 60/905,559, filed Mar. 7, 2007, which is incorporated by reference herein in its entirety.

### FIELD

The present invention relates generally to glucosamine compositions. In a particular instance, disclosed are compositions of physiologically effective amounts of glucosamine for rapid enhancement of at least one of mental performance and physical performance of a subject and methods for use of the same.

### BACKGROUND

Health consciousness is gaining popularity among all individuals. Good health can contribute to, for example, increased and/or improved physical and mental stamina. Stamina is necessary to sustain continuous performance, even for people who are normally involved in strenuous physical activities, such as athletes and sportspersons. Likewise, mental stamina is critical to activities involving mental stresses. In such instances, performance can be improved using mental and physical performance enhancers. The energy used in either physical or mental exertion needs to be replenished rapidly if performance levels are to be maintained. This is true for all facets of the population including children, young adults, men, women, seniors, labor and professional workers, and so on.

Stress comes in several interrelated forms, which is why stress is so potentially harmful to good health and well-being. Stress can be mental and brought on, for example, by a traumatic loss, a demanding working environment or a difficult commitment. Stress can also be physical. The effects of physical stress are readily felt, whether it is by an athlete exhausted after a race, or someone having trouble reaching the next level in a gym workout. Physical stresses can show up as weak or aching muscles, poor quality of sleep, bad circulation and even medical or dental problems. Stress could also be a result of blood loss from the body, such as for example, either during or after a medical procedure or other phenomena.

Mental and/or emotional stress may also leave an individual feeling angry, worried or exhausted. Mental focus may become blurred, there may be trouble concentrating, and memory retention may suffer, leading to an overall effect on the individual. As mental and emotional stress continues, the cumulative strain can contribute to obesity, insomnia, depression, diabetes, and heart disease, as well as other ailments.

Many products are available on the market to overcome fatigue brought about by continuous stress due to mental or physical activities. Currently these products include selective diets, vitamins, and stimulants (such as tea and coffee), to increase an individual's athletic prowess. Additionally, products including gingko biloba can be used for memory enhancement.

Compositions including an amino sugar, such as glucosamine and its derivatives, have been used as a supplement for providing relief to the joints. Such compositions are usually delivered in the form of liquids, tablets or capsules, lotions, lozenges, or patches. These amino sugar compositions, however, are not widely accepted for use to enhance physical performance by overcoming physical stress, other than the stress due to pain in the joints. In this mode of action, it is generally accepted that for beneficial joint effects, long amounts of time (weeks to months) are needed to manifest positive benefits. Apart from various energy drinks cited in the literature which include glucose and its derivatives, amino sugars are widely used as supplements to provide relief for those suffering from osteoarthritis.

Transdermal atheletic patches for overcoming the physical stress in the body have been described. These patches do not disclose a rapid effect of using a transdermal atheletic patch to overcome physical stress effects though.

Similarly, aqueous beverages have been used to mitigate joint pain and overcome stress during physical activity, however, none of these products report the use of glucosamine compositions in overcoming physical or mental stress rapidly, i.e. within minutes of its consumption, to provide benefits such as enhanced performance during the activity itself.

Furthermore, where the art presupposes the use of amino sugars to provide recuperative effects in minutes or hours for cellular stress reduction, such usage involves administering glucosamine in a non-convenient form and during trauma situations only.

Although glucosamine is expected to provide benefits by enhancing/maintaining glycoaminoglycans in cartilaginous tissues in joints over a period of time, it has been surprisingly discovered that glucosamine may be distributed throughout bodily tissues by oral ingestion, thus providing a source of glucosamine for glycosylations within cells experiencing stress effects. In fact, recent work has demonstrated that exogenously provided glucosamine can provide for glycosylation reactions in proteins in cells as a part of response cascades to address negative effects of stress within cells. This discovery provides alternatives to the injection forms demonstrated in the art, which are inconvenient and potentially dangerous for common provision in daily circumstances where enhancement of performance during bodily or mental stress might be desired.

As such, the present invention addresses an important need by providing a composition which mitigates stress and enhances mental performance or physical performance of an individual before the start of the activity, and overcomes the related stress within a few minutes of its consumption.

### SUMMARY

Certain embodiments of the present invention alleviate and overcome many of the above mentioned problems and shortcomings of the present state of the art through the discovery of compositions of glucosamine methods for administering compositions for rapid enhancement of at least one of mental performance and physical performance of a subjecty.

Certain embodiments of the present invention provides methods and compositions for rapidly enhancing mental performance or physical performance by reducing cellular stress effects in the body of the subject. Specifically, the present invention relates to glucosamine compositions and methods for rapidly reducing at least one kind of the cellular stress effects in the body of the subject by the use of the glucosamine compositions.

In an embodiment of the invention, a composition and a method is provided for enhancing mental performance of the subject, by reducing at least one kind of cellular stress effects in the body of the subject.

In another embodiment, the present invention provides a composition and method for increasing memory recall of the subjects, thereby increasing mental performance of the subject.

In another embodiment, the present invention provides a composition and method for enhancing physical performance of the subject, by reducing at least one kind of cellular stress effects in the body of the subject.

In an embodiment of the invention, the glucosamine composition is in the form of a conveniently ingested or absorbed product, including one of ready-to drink beverage, sports drink beverage, energy drink beverage, juice, tea, smoothie, powdered beverage, enhanced water, tablet, hard-shell encapsulated composition, soft gelatin encapsulated composition, gum, candy, chew, tabletted confectionaries, food or supplement bar, snack bar, chocolate, jelly, yogurt, cookie, snack, soup, gelatin, powdered supplement, lotion, nasal spray, transdermal patch, and suppository.

The composition in accordance with an embodiment is in the form of other delivery systems, including lotions, sprays, and transdermal patch means.

### DETAILED DESCRIPTION

In the foregoing and following description, for purposes of explanation only, materials and examples are set forth in order to provide a thorough understanding of the invention. Reference in the specification to “one embodiment” or “an embodiment” means that a particular feature or characteristic described in connection with the embodiment is included in at least one embodiment of the invention. The appearances of the phrase “in an embodiment” in various places in the specification are not necessarily all referring to the same embodiment.

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a beverage” includes a plurality of such beverages and reference to “the food product” includes reference to one or more food products and equivalents thereof known to those skilled in the art, and so forth. Similarly, the word “or” is intended to include “and” unless the context clearly indicates otherwise. Hence “comprising A or B” means including A, or B, or A and B.

Unless otherwise indicated, all numbers expressing quantities of ingredients, temperatures, time periods, and so forth used in the specification and claims are to be understood as being modified by the term “about” whether explicitly stated or not. Accordingly, unless indicated clearly to the contrary, the numerical parameters set forth are approximations.

Unless explained otherwise, all technical and scientific terms used herein have the same meaning as commonly understood to one of ordinary skill in the art to which this disclosure belongs.

The term “administer”, as used herein, means to cause a subject to receive something. Administration of the disclosed food products or beverages supplemented with glucosamine is oral, for example, by ingestion. This includes either self-administration or administration by a third party.

The term “serving”, as used herein, refers to the amount of food or beverage a person or animal would customarily eat at one time or in one sitting. The serving size can often times be found on the Nutrition Facts label on a food or beverage. Serving sizes are also shown on the USDA Food Pyramid. For bulk products, such as breakfast cereal and flour, a serving is usually represented in common household terms, such as cup, tablespoon, teaspoon, or fluid ounce. For products that come in discrete units, such as bread and cookies, a serving size is usually listed as the number of units that constitute a serving, such as three cookies or two slices of bread.

The term “subject”, as used herein refers to living multicellular vertebrate organisms, a category which includes both human and veterinary subjects, for example, mammals, rodents, and birds.

The term “physiologically effective amount”, as used herein, means an amount sufficient to achieve a desired biological effect. In one embodiment, it is an amount that is effective to alleviate or reduce symptoms associated with stress effects. A physiologically effective amount of an active substance such as glucosamine indicates an adequate amount of the active substance which has a significant, externally observable effect, on the subject. Thus, such a physiologically effective amount affects one or more of the stress-related symptoms in the subject without the need for special equipment to determine the effect.

The term “cellular stress effects”, as used herein, refers to stress effects at a cellular level. The stress effects at cellular level are caused by exposure to an adverse environmental condition or conditions that significantly perturbs cellular homeostasis. For example, many types of environmental stresses have been shown to cause deleterious changes in protein conformation and others are known to cause DNA damage. For example, the molecular mechanism of damage to DNA and proteins may be mediated in many cases by stress-induced radical formation and changes in cellular redox state. These cellular stress effects, caused by different kinds of cell stressors are overcome by initiating a response and are directed at re-establishing cellular homeostasis with regard to the particular environmental factor that is perturbed. Cell stressors have been known to affect an individual's physical and mental performance. The stressors include, but are not limited to, physical stress, psychological stress, osmotic stress, thermal stress, nutritional stress, mechanical stress, mental stress, memory recall, stress due to medical procedures, senescence, antibiotics, alcohols, metals, and others.

The present invention relates to compositions of a physiologically effective amount of glucosamine for rapid enhancement of mental performance or physical performance of a subject. The physiologically effective amount of glucosamine composition may be administered in the body of the subject in a variety of ways, such as, but not limited to, orally and topically. The glucosamine composition of the present invention enhances mental performance or physical performance by reducing at least one kind of cellular stress effects in the subject's body.

In an embodiment of the present invention, the glucosamine composition is comprised of glucosamine derivatives and glucosamine salts.

Glucosamines are amino sugars or molecules derived from amino acids and sugar. Naturally found in the body, glucosamine is an essential component of glycosaminoglycans, substances which are essential in restoring damaged cartilage and reducing inflammation. As a supplement, glucosamine's principal use has been for relief of osteoarthritis, not only for symptomatic relief, but to help the body maintain existing or possibly replace lost cartilage. The present invention describes rapid enhancement of mental performance or physical performance by reducing the cellular stress effects in the subject's body by administering a physiologically effective amount of a glucosamine composition.

For example, within the scope of this invention are glucosamine containing compositions that include glucosamine sulphate, glucosamine hydrochloride, n-acetyl glucosamine, glucosamine hydroiodide, or other salts form, derivatives, mixtures or combinations thereof.

Glucosamine can be prepared by various methods. Glucosamine is primarily derived from harvested natural sources, such as shellfish and other aquatic organisms. Non limiting examples of methods of preparing glucosamine of the present invention is also provided in U.S. application Ser. No. 10/326,549 and related family members, which is incorporated herein by reference.

As stated previously, the glucosamine composition may be conveniently administered in a variety of ways, including orally and topically.

The orally administered embodiments of the glucosamine composition may be in the form of a conveniently ingested product, including one of ready-to drink beverage, sports drink beverage, energy drink beverage, juice, tea, smoothie, powdered beverage, enhanced water, tablet, hard-shell encapsulated composition, soft gelatin encapsulated composition, food supplement. Other embodiments include food supplements which can be in the form of gum, candy, chew, tabletted confectionaries, food or supplement bar, snack bar, chocolate, jelly or gel, yogurt, cookie, snack, soup, gelatin, and powdered supplements. The non edible form of the composition may be administered in the form of lotions, sprays, nasal spray, suppositories and transdermal patches.

In an embodiment, the glucosamine composition can include crystallized glucosamine that may be used conveniently to prepare beverages, e.g., tea or juice. The crystallized glucosamine can also be used to prepare paste, jelly, capsules, or tablets. In an embodiment of the invention, lactose and corn starch may be used as diluents for capsules and as carriers for tablets.

The composition of this invention can also be a dietary supplement, cosmetic, or a pharmaceutical formulation. The composition can also be a drink or food product. The composition could also be a pet or animal food or veterinarian product. As used herein, the terms “drink” and “food” broadly refer to any kinds of liquid and solid/semi-solid materials, respectively, that are used for nourishing an animal, including a human, and for sustaining normal or accelerated growth of the animal. Examples of the drink product include, but are not limited to, tea-based beverages, juice, coffee, and milk. Examples of the food product include jelly, cookies, cereals, chocolates, snack bars, herbal extracts, dairy products (e.g., ice cream, and yogurt), soy bean product (e.g., tofu), and rice products.

In an embodiment of the invention, the glucosamine composition may be combined with a physiologically acceptable oral vehicle into unit dosages. A unit dosage may comprise a physiologically effective amount of glucosamine composition for a single administration (e.g orally). A unit dosage may depend on one or more factors including age, size, and condition of the subject being treated, as well as the number of times the unit will be taken in a single day.

In an embodiment of the present invention, the glucosamine composition contains an amount of glucosamine between 250 mg and 5000 mg, or between 500 mg and 5000 mg to be administered per day. In an embodiment of the invention, when the composition is administered orally, the serving size is from 500 mg to 1500 mg or between 250 mg and 1500 mg.

Composition in the form of formulations for topical administration may include but are not limited to lotions, ointments, gels, creams, suppositories, drops, liquids, sprays, patches, and powders.

Oral administration is accomplished by injesting the composition.

The amount of glucosamine utilized should be determined appropriately for the product (i.e. capsule, food bar, etc.) being created.

In one embodiment of the present invention, the physiologically effective amount is the amount that is effective in alleviating or reducing the symptoms of physical stress effects, such as by increasing the oxygen delivery to the tissues in the body. In particular examples, it is a concentration of glucosamine composition that is effective to alleviate, reduce, or stabilize symptoms associated with a physical stress effect, alone or in combination with other agent, such as in an individual subject to whom the glucosamine composition is administered. In one embodiment, it is an amount that is effective to alleviate or reduce symptoms of physical stress effects, such as increasing the ability of the body to recover rapidly from blood loss from the body. In other examples, it is a concentration of glucosamine composition that is effective to alleviate, reduce, or stabilize symptoms associated with a physical stress effects, alone or in combination with other agent, in an individual subject to whom glucosamine composition is administered.

In one embodiment of the present invention, it is an amount that is effective to alleviate or reduce symptoms of mental stress effects, such as increasing the mental performance of the subject. In particular embodiments it is a concentration of glucosamine composition that is effective to alleviate, reduce, or stabilize symptoms associated with a mental stress effect alone or in combination with other agent or agents, in an individual subject to whom the glucosamine composition is administered.

In one embodiment, the physiologically effective amount also includes a quantity of glucosamine sufficient to achieve a desired effect in the subject being treated. For instance, it can be an amount necessary to increase the focus and concentration to speed up the responding and decision making ability of the subject. In particular examples, it is a concentration of glucosamine composition that is effective to increase the focus and concentration to speed in responding and decision making, alone or in combination with other agent or agents, in an individual subject to whom the glucosamine composition is administered.

The present invention provides one such composition which rapidly reduces cellular stress effects in the subject's body, thereby improving physical and mental performance.

Cellular stress effects are caused by various factors. Various stressors tend to affect a body's performance. Physical stressors may include, but are not limited to, heat, cold, several types of irradiation, including ultra violet light and magnetic fields. Psychological stressors may include, but are not limited to, emotions, aging, emotional conflicts, and hormonal imbalance. Mechanical stressors may include, but are not limited to, compression, shearing, and stretching. Apart from these, there are other kinds of cellular stressors such as those relating to oxygen, pH, biologic, osmotic, nutritional, stress due to antibiotics, alcohols, metals, and others. Athletic events such as exercising, running, sports related activities, and strength training, can also cause stress in the body, thus reducing the performance. Activities such as examinations, strenuous work, public speaking, and traveling can also induce stress in the body.

The body naturally overcomes some of these stresses by activating stress genes. However, the body's natural ability to overcome stress can be limited and at the same time slow, in which case taking nutritional supplements from an external source enhances the body's recovery from stress and improves performance.

As used herein, rapid reduction of cellular stress effects refers to the time in which a desired effect of the physiologically effective amount is observed in the body of the subject. The desired effect refers to improving the mental or physical performance of the subject's body within about 30 minutes of administering the amount.

In one embodiment of the invention, physical performance of the subjects is increased by one to one hundred percent using the physiologically effective amount. This increase in physical performance is due to reduction in physical stress effects, which is reduced from 1 to 85 percent.

In another embodiment of the present invention, mental performance of subjects is increased by one to eighty percent using a physiologically effective amount of the composition. This increase in mental performance is due to reduction in mental stress effects, which may be reduced from 1 to 60 percent. In another embodiment, the memory of the subject may be increased from 1 to 99 percent.

The above-described composition, in any of the forms described above, can be used for enhancing physical performance. As shown in examples below, the composition enhances overall strength, balance, fatigue recovery, intensity of physical exercise, muscle relaxation, and endurance for exercise. The terms “improving”, “enhancing”, “treating,” and “lowering” refer to the administration of a physiologically effective amount of a composition of the invention to a subject in need of physical performance improvement, with the purpose to improve physical performance before or during the start of any physical activity, but do not imply treatment of a disease.

The above-described composition, in any of the forms described above, can be used for enhancing mental performance. As shown in examples below, the composition enhances overall mental performance of the subject. Mental stress can make a subject anxious, nervous, worried, tired, or strained for prolonged periods of time without relief. As shown in examples below, the composition overcomes mental stress effects such as inability to focus, reduced concentration of energy, and weak memory skills. The terms “improving”, “enhancing”, “treating,” and “lowering”, “reducing” refer to the administration of an effective amount of the composition of the invention to the subject who needs to enhance his mental performance, with the purpose to improve mental performance before or during the start of any mental activity, but do not imply the treatment of a disease.

In one of the embodiments, mental performance relates to improving the memory skills of the subject. Memory is the retention of information over time. The most popularly studied kind of memory is recall. Memory recall is related to improving both short term and long term memory of the subject.

As used herein, short term memory refers to recalling the information heard and seen a few seconds earlier, and lasts for more than a few seconds to a minute. As used herein, long term memory refers to the memory that lasts from a minute or so to weeks or even years.

According to an embodiment, the method of the present invention includes enhancement of mental performance or physical performance by administering the glucosamine composition as described above within a timeframe of five hours before to five hours after the start of an event involving cellular stress. In certain embodiments, a method of the present invention includes enhancement of mental performance or physical performance by administering the glucosamine composition as described above within a timeframe of 30 minutes before the event involving cellular stress.

The event according to present invention includes, but is not limited to, exercising, running, sports related activities, strength training, exam taking, work stressors, public speaking, hyperthermia, and hypothermia.

The event, according to one embodiment, includes blood loss. Loss of blood from the body of the subject may occur during one of the following activities: blood donation, menstruation, medical procedure, or injury. Medical procedure according to the present invention includes a procedure employed by a medical or dental practitioner. This may further include surgical operations, surgical procedures, surgical processes, surgery, operations, dental procedures, and the like.

The following non-limiting examples are given by way of illustration only and are not to be considered limitations of this invention. There are many apparent variations within the scope of this invention.

### Example 1

### Effect on Reducing Mental Stress Effects

Composition A was prepared by mixing 1500 mg glucosamine (glucosamine hydrochloride) in V8® splash, obtained from the Campbell Soup Company, to constitute one serving. One serving is 8 ounces.

Before the subjects were selected they were asked the following questions:
Have you taken any glucosamine supplement today?
Do you have any serious heart problems or epilepsy and the like?
The subject was selected only if the subject was willing to consume glucosamine and the answer to both the questions was ‘no’.

Twelve adult subjects of ages ranging from 21 to 59 years were classified into two groups of six each i.e. a test group and a control group (placebo group). The test group received 1500 mg of glucosamine in V8® splash and the control group received only V8® splash, and this was ingested 30 minutes prior to the stress event.

The subjects were asked to take a mental test. They were asked to sort a set of 25 word cards in alphabetical order. The results of the test are shown in Table 1. During this test the stress was induced by alarm buzzers, strobe light, and other stimulants.

### Table 1. The Effectiveness of the Glucosamine Composition in Reducing the Mental Stress Effects of a Subject

TABLE 1
Time to sort the cards (in secs)
Test Group (n = 6)
113 ± 22.4
Control Group (n = 6)
140.2 ± 21.7

Table 1. The control group took average of 140.2 seconds to sort the cards as compared to the test group which took significantly lesser time, of an average of 113 seconds for sorting the cards. Thus, Table 1 shows that consumption of composition A significantly reduced the time to sort the cards.

Also, as is evident from Table 2a as well, the mental stress effects of the test group was reduced as a whole compared with the placebo group. Accordingly, the tests show that the glucosamine composition reduces mental stress effects. The reduction in mental stress effects improved the mental performance of the subjects.

Table 2a. The Effectiveness of the Glucosamine Composition in Increasing the Memory Recall of a Subject

TABLE 2a
Number of Words Remembered
Test Group (n = 6)
6.2 ± 3.1
Control Group (n = 6)
5.8 ± 1.2

### Example 2

### Effect on Increasing Memory

The test was initially performed without the instructions to remember the words as shown in Phase I.

### Phase 1: Without Instructions to Remember the Words

Composition A was prepared by mixing 1500 mg glucosamine (glucosamine hydrochloride) in V8® splash, obtained from the Campbell Soup Company, to constitute one serving. One serving is 8 ounces.

Before the subjects were selected they were asked the following questions:
Have you taken any glucosamine supplement today?
Do you have any serious heart problems or epilepsy and the like?
The subject was selected only if the subject was willing to consume glucosamine and the answer to both the questions was ‘no’.
Twelve adult subjects of age ranging from 21 to 59 years old were classified into two groups of six each, i.e., a test group and a control group (placebo group). Thirty minutes prior to the stress event, the test group received 1500 mg of glucosamine in V8® splash and the control group received only V8® splash

The subjects were asked to take the memory test by asking the subjects to recall all the words which they were shown during the mental test. The mental test was the same as in Example 1. The results of the test are shown in Table 2a. Stress was induced in the subjects by alarm buzzers, strobe light, and other stimulants.

Table 2a. Control group could recall an average of 5.8 words as compared to test group, which could recall an average of 6.2 words.

The results show an improvement in the test group that was given the glucosamine. The effect of glucosamine on memory enhancement was even more apparent with the group that was instructed to remember the words as shown in Phase II below.

### Phase II: With Instructions to Remember the Words

Composition A was prepared by mixing 1500 mg glucosamine (glucosamine hydrochloride) in V8® splash, obtained from the Campbell Soup Company, to constitute one serving. One serving is 8 ounces.

Before the subjects were selected they were asked the following questions:
Have you taken any glucosamine supplement today?
Do you have any serious heart problems or epilepsy and the like?
The subject was selected only if the subject was willing to consume glucosamine and the answer to both the questions was ‘no’.
Eight adult subjects of age ranging from 21 to 53 years old were classified into two groups of four each i.e. a test group and a control group (placebo group). The test group received 1500 mg of glucosamine in V8® splash and the control group received only V8® splash 30 minutes prior to the stress event.

The subjects were asked to take the memory test, by asking the subjects to recall all the words which they were shown during the mental test. The mental test was the same as shown in Example 1, except that subjects were not subjected to stress such as alarm buzzers, strobe light, and other stimulants. Also, the subjects were informed that they would be undergoing a memory test. The results of the test are shown in Table 2b.

Table 2b. Control group could recall average 5.5 words as compared to test group which could recall, of an average of 8.5 words. It was concluded from the experiment that glucosamine significantly enhanced the memory as the difference between test and control group was statistically significant.

Table 2b. The Effectiveness of the Glucosamine Composition in Increasing the Memory Recall of a Subject

TABLE 2b
Number of Words Remembered
Test Group (n = 4)
8.5 ± 1.29
Control Group (n = 4)
5.5 ± 0.58

### Example 3

### Physical Stress Effects Test

Composition A was prepared by mixing 1500 mg glucosamine (glucosamine hydrochloride) in V8®, obtained from the Campbell Soup Company to constitute one serving. One serving is 8 ounces.

Before the subjects were selected they were asked the following questions:
Have you taken any glucosamine supplement today?
Do you have any serious heart problems or epilepsy and the like?
The subject was selected only if the subject was willing to consume glucosamine and the answer to both the questions was ‘no’.
Twelve adult subjects of age ranging from 21 to 59 year old were classified into two groups of six each i.e. a test group and a control group (placebo group). The test group received 1500 mg of glucosamine in V8® splash and the control group received only V8® splash 30 minutes prior to the stress event.

The subjects were asked to take the physical test, by asking the subjects to do a stair stepping activity. The results of the test are shown in Table 3. The heart beat rate was measured before the start of the physical test and at the end of the physical test. The difference in the heart rate would indicate reduction in stress effects

Table 3. The average change in the heart beat rate in control group was found to be 18.7, while the test group showed a change of 22.7. The percentage increase in the heart beat rate of the control group was 26 percent, while the test group measured a 29 percent increase. The increased heart beat rate in the glucosamine-treated subjects results in reduction of physical stress effects by better oxygen delivery to the tissue.

### Table 3 The Effectiveness of the Glucosamine Composition in Increasing the Physical Performance of a Subject

TABLE 3
Change in Heart Rate
Test Group (n = 6)

| | |
|---|---|
| Before ingestion | 74.67 ± 14.01 |
| After ingestion | 97.33 ± 18.01 |
| Change | 22.70 ± 6.0 |

Control Group (n = 6)

| | |
|---|---|
| Before | 73.33 ± 7.45 |
| After | 92.00 ± 9.12 |
| Change | 18.70 ± 9.12 |

Certain embodiments of the invention comprise the following:

A composition comprising a physiologically effective amount of glucosamine, for rapid enhancement of at least one of mental performance and physical performance of a subject. In this embodiment the glucosamine may comprise glucosamine salts and derivatives. In certain embodiments the glucosamine in this composition comprises at least one of N-acetyl glucosamine, glucosamine hydrochloride, glucosamine sulphate, glucosamine hydroiodide, glucosamine organic acids, and glucosamine phosphate.

Another embodiment is a composition comprising a physiologically effective amount of glucosamine, for rapid enhancement of at least one of mental performance and physical performance of a subject, wherein the rapid enhancement of at least one of mental performance and physical performance comprises reduction of cellular stress effects. In another embodiment this composition includes the cellular stress comprising at least one of physical stress, psychological stress, and mechanical stress.

Another embodiment is a composition comprising a physiologically effective amount of glucosamine, for rapid enhancement of at least one of mental performance and physical performance of a subject, wherein the rapid enhancement of at least one of mental performance and physical performance comprises reduction of cellular stress effects, wherein the cellular stress is a result of at least one of exercising, running, sports related activities, strength training, weight training, taking an exam, work stressors, public speaking, traveling, hyperthermia, and hypothermia.

Another embodiment is a composition comprising a physiologically effective amount of glucosamine, for rapid enhancement of at least one of mental performance and physical performance of a subject, wherein the physical performance is enhanced by 1 to 100 percent.

Another embodiment is a composition comprising a physiologically effective amount of glucosamine, for rapid enhancement of at least one of mental performance and physical performance of a subject, wherein the mental performance is enhanced by 1 to 80 percent.

Another embodiment is a composition comprising a physiologically effective amount of glucosamine, for rapid enhancement of at least one of mental performance and physical performance of a subject, wherein the rapid enhancement of mental performance comprises improving memory recall of the subject by 1 to 100 percent/units. Certain embodiments of this composition the memory recall of the subject comprises at least one of long-term memory and short-term memory.

Another embodiment is a composition comprising a physiologically effective amount of glucosamine, for rapid enhancement of at least one of mental performance and physical performance of a subject, wherein the composition comprises the glucosamine in an amount less than or equal to 5 grams per serving. Another embodiment is a composition comprising a physiologically effective amount of glucosamine, for rapid enhancement of at least one of mental performance and physical performance of a subject, wherein the composition comprises the glucosamine in an amount of 0.5 to 1.5 grams per serving.

Another embodiment is a composition comprising a physiologically effective amount of glucosamine, for rapid enhancement of at least one of mental performance and physical performance of a subject, wherein the composition is in the form of an ingestion product, the ingestion product comprising at least one of ready-to drink beverage, sports drink beverage, energy drink beverage, juice, tea, smoothie, powdered beverage, enhanced water, tablet, hard-shell encapsulated composition, soft gelatin encapsulated composition, food supplement, soup, gelatin, and powdered supplement. In another embodiment the food supplement comprises at least one of gum, candy, chew, tabletted confectionaries, food or supplement bar, snack bar, soup, chocolate, jelly or gel, gelatin, yogurt, cookie, snack and powdered supplement.

Another embodiment is a composition comprising a physiologically effective amount of glucosamine, for rapid enhancement of at least one of mental performance and physical performance of a subject, wherein the composition comprises one of a lotion, spray, suppository, or a transdermal patch.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the event involving cellular stress comprises at least one of exercising, running, sports related activities, strength training, weight training, taking an exam, work stressors, public speaking, traveling, hyperthermia, and hypothermia.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the event includes a blood loss event. In another embodiment of this method the blood loss event comprises at least one of blood donation, menstruation, medical procedure, and injury.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the glucosamine comprises glucosamine salts and derivatives.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the glucosamine comprises at least one of N-acetyl glucosamine, glucosamine hydrochloride, glucosamine sulphate, glucosamine hydroiodide, glucosamine organic acids, and glucosamine phosphate.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the cellular stress effects comprises at least one of physical stress, psychological stress, and mechanical stress.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the physical performance is enhanced by 1 to 100 percent.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the mental performance is enhanced by 1 to 80 percent/units.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the rapid enhancement of mental performance comprises improving memory recall of the subject by 1 to 100 percent/units. In another embodiment of this method, the memory recall of the subject comprises at least one of long-term memory and short-term memory.

Another embodiment of the invention is a method for rapid enhancement of at least one of mental performance and physical performance of a subject, the method comprising administering to the subject a physiologically effective amount of glucosamine within a time frame of five hours before to five hours after the start of an event involving cellular stress effects, wherein the glucosamine is administered at 30 minutes prior to the event involving the cellular stress.

In view of the many possible embodiments to which the principles of this disclosure may be applied, it should be recognized that the illustrated embodiments are only particular examples of the disclosure and should not be taken as a limitation on the scope of the disclosure. Rather, the scope of the disclosure is in accord with the following claims.

What is claimed is:

1. A composition comprising a physiologically effective amount of glucosamine, for rapid enhancement of at least one of mental performance and physical performance of a subject.

2. The composition of claim 1, wherein the glucosamine comprises glucosamine salts and derivatives.

3. The composition of claim 1, wherein the glucosamine comprises at least one of N-acetyl glucosamine, glucosamine hydrochloride, glucosamine sulphate, glucosamine hydroiodide, glucosamine organic acids, and glucosamine phosphate.

4. The composition of claim 1, wherein the rapid enhancement of at least one of mental performance and physical performance comprises reduction of cellular stress effects.

5. The composition of claim 4, wherein the cellular stress comprises at least one of physical stress, psychological stress, and mechanical stress.

6. The composition of claim 4, wherein the cellular stress is a result of at least one of exercising, running, sports related activities, strength training, weight training, taking an exam, work stressors, public speaking, traveling, hyperthermia, and hypothermia.

7. The composition of claim 1, wherein the physical performance is enhanced by 1 to 100 percent.

8. The composition of claim 1, wherein the mental performance is enhanced by 1 to 80 percent.

9. The composition of claim 1, wherein the rapid enhancement of mental performance comprises improving memory recall of the subject by 1 to 100 percent/units.

10. The composition of claim 9, wherein the memory recall of the subject comprises at least one of long-term memory and short-term memory.

11. The composition of claim 1, wherein the composition comprises the glucosamine in an amount less than or equal to 5 grams per serving.

12. The composition of claim 11, wherein the composition comprises the glucosamine in an amount of 0.5 to 1.5 grams per serving.

13. The composition of claim 1, wherein the composition is in the form of an ingestion product, the ingestion product comprising at least one of ready-to drink beverage, sports drink beverage, energy drink beverage, juice, tea, smoothie, powdered beverage, enhanced water, tablet, hard-shell encapsulated composition, soft gelatin encapsulated composition, food supplement, soup, gelatin, and powdered supplement.

14. The composition of claim 13, wherein the food supplement comprises at least one of gum, candy, chew, tabletted confectionaries, food or supplement bar, snack bar, soup, chocolate, jelly or gel, gelatin, yogurt, cookie, snack and powdered supplement.

15. The composition of claim 1, wherein the composition comprises one of a lotion, spray, suppository, or a transdermal patch.

16. A method for rapid enhancement of at least one of mental performance and physical performance of a subject, the method comprising administering to the subject a physiologically effective amount of glucosamine within a time frame of five hours before to five hours after the start of an event involving cellular stress effects.

17. The method of claim 16, wherein the event involving cellular stress comprises at least one of exercising, running, sports related activities, strength training, weight training, taking an exam, work stressors, public speaking, traveling, hyperthermia, and hypothermia.

18. The method of claim 16, wherein the event includes a blood loss event.

19. The method of claim 18, wherein the blood loss event comprises at least one of blood donation, menstruation, medical procedure, and injury.

20. The method of claim 16, wherein the glucosamine comprises glucosamine salts and derivatives.

21. The method of claim 16, wherein the glucosamine comprises at least one of N-acetyl glucosamine, glucosamine hydrochloride, glucosamine sulphate, glucosamine hydroiodide, glucosamine organic acids, and glucosamine phosphate.

22. The method of claim 16, wherein the cellular stress effects comprises at least one of physical stress, psychological stress, and mechanical stress.

23. The method of claim 16, wherein the physical performance is enhanced by 1 to 100 percent.

24. The method of claim 16, wherein the mental performance is enhanced by 1 to 80 percent/units.

25. The method of claim 16, wherein the rapid enhancement of mental performance comprises improving memory recall of the subject by 1 to 100 percent/units.

26. The method of claim 25, wherein the memory recall of the subject comprises at least one of long-term memory and short-term memory.

27. The method of claim 16, wherein the glucosamine is administered at 30 minutes prior to the event involving the cellular stress.
